(19) United States
(12) Patent Application Publication
(10) Pub. No.: US 2025/0001061 A1
(43) Pub. Date: Jan. 2, 2025

(54) DIALYSIS DEVICE WITH CONNECTION UNIT

(71) Applicant: FRESENIUS MEDICAL CARE DEUTSCHLAND GMBH, Bad Homburg (DE)

(72) Inventors: Jurgen HACKER, Bad Homburg (DE); Oliver BOND, Bad Homburg (DE); Markus KOHLER, Bad Homburg (DE)

(73) Assignee: FRESENIUS MEDICAL CARE DEUTSCHLAND GMBH, Bad Homburg (DE)

(21) Appl. No.: 18/707,181

(22) PCT Filed: Nov. 8, 2022
(86) PCT No.: PCT/EP2022/081041

§ 371 Date: May 3, 2024

(30) Foreign Application Priority Data
Nov. 9, 2021 (DE) 10 2021 129 109.7

(51) Int. Cl. A61M 1/36; A61M 39/10

(52) U.S. Cl. CPC A61M 1/362266; A61M 39/1011; A61M 2205/15

(57) ABSTRACT

The invention relates to a medical treatment device with a housing, with a connection unit for producing a fluidic connection between the hydraulics of the treatment device and a connector connected to a disposable that conducts fluid during use. This connection is used to convey dialysate into the extracorporeal blood circuit. The connection unit and the connector are designed such that particularly hygienic conditions exist upon connection and disconnection of the disposable.

### TECHNICAL FIELD

The invention relates to a machine for extracorporeal blood treatment with a connection unit which can be used to supply dialysate to the extracorporeal blood circuit.

### BACKGROUND

In extracorporeal blood treatments, for example in chronic haemodialysis, it may be advantageous, during the treatment, to add the treatment solution, in particular the dialysis solution, to the blood that is to be treated. For this purpose, one or more connection units are made available on the treatment devices, and lines are attached to the connection units in order to lead liquids out of the dialysate-carrying hydraulics of the dialysis machine and feed them to the extracorporeal blood circuit. Lines that can be discarded after use are referred to as disposables. In most systems, the connection of the blood hose system to this connection unit is established by the operator. The connection unit is located, together with the blood hose system, on the front of the appliance. This connection unit of the treatment machine establishes a direct connection of the machine interior to the disposable and in particular also to the blood circulation of the patient, without a filter being arranged between blood circulation and machine hydraulics. This connection unit has to satisfy strict hygiene requirements in order to minimize the risk of microbes getting into this interface from the outside.

The object of the present invention is to make available a medical treatment device with a suitable connection unit, a suitable disposable connector, and a system consisting of a medical treatment device and a connector.

### SUMMARY OF THE INVENTION

According to the teaching of the present invention, this object is achieved by a medical treatment device according to claim 1, a disposable according to claim 11 and a system according to claim 13.

Particular embodiments are the subject matter of the dependent claims.

The invention relates to a medical treatment device with a housing and with a connection unit that protrudes through a wall of the housing for producing a fluidic connection between at least a first fluid line assigned to the interior of a treatment device and a connector connected to a second fluid line, e.g. a fluid line of a disposable that conducts fluid during use. The second fluid line does not count as part of the treatment device and is instead assigned to the exterior of the treatment device. Fluid is to be understood in particular as a liquid, e.g. a dialysis treatment solution. The fluid line of the disposable can be, for example, a hose set or part thereof, a substituate line or the like.

The housing has walls, which enclose the interior of the treatment machine.

The connection unit has an outer tube and an inner tube, which is arranged in the lumen of the outer tube. A lumen within the meaning of the invention is an inner cavity of a hollow body, here of the outer tube of the connection unit. The inner tube can be arranged concentrically in the lumen of the outer tube. The inner tube can have an inner lumen that tapers conically in the direction from the exterior to the interior of the treatment device.

The outer tube has a first outer-tube opening at the end facing the exterior of the treatment device and at least a second and third outer-tube opening at the end facing the interior of the treatment device.

The inner tube has a first inner-tube opening at the end facing the exterior of the treatment device and at least a second inner-tube opening at the end facing the interior of the treatment device.

The outer tube protrudes permanently out of the housing of the treatment device, such that the first outer-tube opening lies in a plane in front of the housing.

Here, permanently means that the outer tube, particularly during connection and disconnection procedures, can be displaced in relation to the wall along the longitudinal axis by 0.1-1.5 mm, e.g. to compensate for dimensional tolerances of the disposable. However, at no time does the outer-tube opening directed to the machine exterior lie on a plane or behind the wall of the housing.

Embodiments according to the invention can have one or more of the features mentioned below, in any technically possible combination.

Wherever spatial references such as “top”, “bottom”, “upper” or “lower” are mentioned herein, a person skilled in the art will if in doubt understand these as a spatial indication relative to the orientation in the appended figures and/or to the arrangement of the port according to the invention during its intended use.

The connector can be a disposable connector; however, the connector can also be used a number of times.

In one embodiment, the outer tube can protrude through a wall of the housing.

The distance, along the longitudinal axis of the connection unit, between the first outer-tube opening and the wall of the housing can measure at least 10 mm.

In relation to the outer-tube opening, the inner-tube opening can be set back in the direction of the machine interior. The distance, along the longitudinal axis of the connection unit, between inner-tube opening and outer-tube opening can thus measure at least 5 mm, preferably 6 mm. Thus, with an outer-tube opening having a diameter of 11 mm, it is possible to prevent accidental contact with non-sterile surfaces during connection and disconnection procedures.

The outer tube can be arranged spaced apart from the wall, such that a wall of the connector can be received in the resulting gap. Preferably, it can be received sealingly through the arrangement of sealing elements in the wall or the outer tube.

The connection unit can be arranged in the treatment machine in such a way that the longitudinal axis of the connection unit is arranged substantially perpendicularly, which means e.g. 80-100° to the wall of the housing. The connection unit, in particular the outer tube of the connection unit, can be fastened in an axially resilient manner to the housing or the wall by a fastening means. The fastening means can have a spring force of 20-40N. The fastening means can be a helical spring. This resilient fastening means can ensure that, even with certain dimensional tolerances of the disposable or the connection unit, it is possible to produce a secure, tight connection between the two components and to prevent leakages that present a risk to hygiene.

For the secure connection of the disposable, thread means can also be provided in the wall, which thread means are provided together on the disposable.

The connection unit can have a leakage sensor, in particular a conductivity sensor, in the lumen between outer tube and inner tube.

The leakage sensor preferably protrudes into the second lumen, i.e. it issues from a side wall and ends free. The leakage sensor is preferably a pin or ring or has a pin or ring.

In some embodiments, the leakage sensor carries or comprises an insulating layer or galvanic isolation. Particularly if this protrudes from a wall to which the leakage sensor is connected, it can prevent incorrect measurements caused by residual moisture.

The leakage sensor can detect any loss of leaktightness between the inner tube of the connection unit and the connector of the disposable and can output a signal. A contamination of the connection unit, potentially occurring as a result of the loss of leaktightness, can then be rectified by initiating a disinfection of the machine hydraulics.

In the state in which the connection unit is not connected to the disposable, it can be closed by a closure element at that end of the outer tube directed to the machine exterior.

The closure element can have a cylindrical element, preferably of stainless steel, which in the closure state protrudes into the outer tube of the connection unit.

An elastic sealing element can be mounted on the inner surface of that end of the outer tube directed to the exterior of the treatment device, preferably being pressed firmly into the outer tube, which elastic sealing element forms a radial form-fit seal with the cylindrical element of the closure element.

The closure element can be provided in a receiving portion of a pivot lever which, for opening and closing the connection unit, is displaceable in translation along a pivot axis, preferably arranged perpendicular to the wall, and wherein the treatment device has a damping device for slowing this translational movement.

On the side directed to the housing front, the pivot lever can have a peripheral moulded seal which prevents liquids or particles from penetrating into the receiving portion of the pivot lever.

Before and/or between the dialysis treatments, the hydraulics of the treatment machine are disinfected and/or flushed. Before the closure element is opened for connection to the disposable, the flushing solution is pumped off from the connection unit and replaced by air. To avoid microbial transfer, it is sought to remove the liquid as completely as possible. For this reason, the lumen between outer tube and inner tube can narrow in the direction of the machine interior in a tapering manner, preferably in a conically tapering manner. Improved removal of the liquid is thus achieved when emptying the port.

The fastening of the connection site can take place such that the longitudinal axis of the connection site is inclined by 5° or more with respect to a set-down surface for the medical treatment device, in such a way that at least one of the outer-tube openings directed to the machine interior lies lower than the outer-tube opening directed to the machine exterior. In this way too, the complete emptying of the flushing solution is improved.

The treatment device can furthermore have a sensor for detecting the state of connection of the connection unit to the connector of the disposable.

The treatment device can be a device for extracorporeal blood treatment.

The invention also comprises a disposable with a fluid line and with a connector with a connector lumen for fluidically connecting the fluid line to the inner tube of a connection unit of a medical treatment device according to one of claims 1 to 10.

The connector has a touch-guard sleeve with a free connection opening and a wall. The connector lumen, surrounded by the wall, leads into an opening that is set back in relation to the free connection opening of the touch-guard sleeve. By means of this embodiment, it is possible to reduce the risk of transfer of microbes which, possibly through collision with non-sterile surfaces or by touching touchable surfaces, could enter the line system of the treatment machine during the connection or disconnection of the disposable. The wall of the touch-guard sleeve has a radially outwardly directed sealing means. It is thereby possible to prevent or reduce the penetration of liquids, located on the exterior of the housing, into the housing interior.

Furthermore, the wall can have means for producing a threaded closure, e.g. cams, such that no accidental loosening of the disposable can take place during the treatment. The locking on the housing surface saves the need for locking means in the connection unit, such that better and/or easier cleaning is possible.

For more convenient connection of the connector, the connector can have a cylindrical part, for connecting to the connection site, and a square grip part.

The invention comprises a system composed of a medical treatment device according to one of claims 1 to 10 and of a disposable according to either of claims 11 and 12.

The system can be designed in such a way that the outer tube of the connection unit is inserted into the lumen of the touch-guard sleeve, and the connector lumen of the disposable is inserted into the lumen of the inner tube of the connection unit. A leaktight fluid line is produced by the conical tapering of the cross section of the inner tube.

Furthermore, the connection between connection unit and connector can be secured by thread means on the outer surface and on the housing or the wall of the medical treatment device.

A plug-in force of between 20 and 40 N for the disposable is set by the resilient fastening of the connection unit, as a result of which easy disconnection by the operator is ensured.

### BRIEF DESCRIPTION OF THE DRAWINGS

FIG. 1: Schematic view of the medical treatment device

FIG. 2: Schematic view of the machine-side end of the connection unit with the section planes A and B

FIG. 3: Schematic view of the longitudinal section through a plane A of the connection unit with closure element

FIG. 4: Schematic view of the longitudinal section through a plane B of the connection unit according to the invention with plugged-in connector

FIG. 5: Schematic view of a leakage sensor according to the invention

FIG. 6: Schematic view of the pivot lever with damper

FIG. 7: Longitudinal section through connector

FIG. 8: Plan view of connector

### DETAILED DESCRIPTION OF AN ILLUSTRATIVE EMBODIMENT

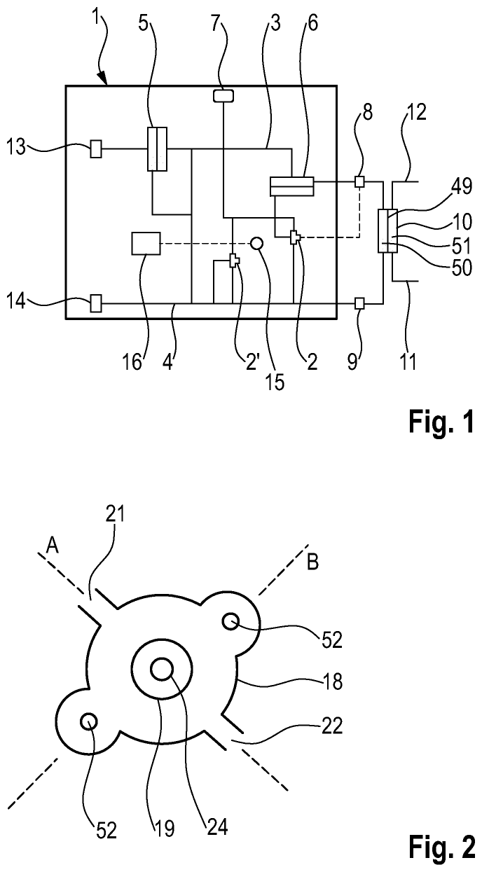

FIG. 1 shows a highly simplified schematic view of an extracorporeal blood treatment device 1 as an example of a medical treatment device.

The extracorporeal blood treatment device 1 is a haemo(dia)filtration device, which is connected to a haemofilter 10. The haemofilter 10 is divided by a semipermeable membrane 49 into a blood chamber 51 and a dialysis liquid chamber 50, through which dialysis liquid flows. The blood chamber 51 is a part of an extracorporeal blood circuit, while the dialysis liquid chamber 50 is connected to the dialysis liquid system of the blood treatment device.

The extracorporeal blood circuit comprises an arterial blood line 11, which leads to the blood chamber 51, and a venous blood line 12, which issues from the blood chamber 51 of the blood filter.

The fresh dialysis liquid is made available in a dialysis liquid source 13. From the dialysis liquid source 13, a dialysis liquid supply line 3 leads to a first sterile filter 5, whose retentate is routed directly to the dialysis discharge line 4. The permeate of the sterile filter 5 is routed to a second sterile filter 6. The retentate of this second sterile filter is routed through the dialysis liquid supply line 3 to the fresh dialysate coupling 8 and from there into the dialysis liquid chamber 50 of the haemofilter 10. After passage through the haemofilter, the discharge of the haemofilter is fed to the dialysate drain 14 via the discharge dialysate coupling 9 into the dialysate discharge line 4 of the machine hydraulics.

The permeate of the sterile filter 6 is conveyed to the connection unit 2 and can be conveyed from there directly into the extracorporeal blood circuit via a disposable line with connector (not shown).

For as long as it is not connected to the disposable, the connection unit 2 is closed by a pivot lever 28 with closure means 17 (not shown here). The blood treatment device has a sensor 15. The latter can detect the correct connection, or the position of the pivot lever, in the closure state of the connection unit, or with the connection unit opened, and report to a control unit.

FIG. 2 shows a schematic view of the machine-side end of the connection 2, 2′, with the outer tube 18 and the second and third outer-tube opening, the inner tube 19 and the second inner-tube opening 24, and the fastening means 52 for fixing the connection unit 2, 2′ in the machine housing, illustrating the section planes A and B.

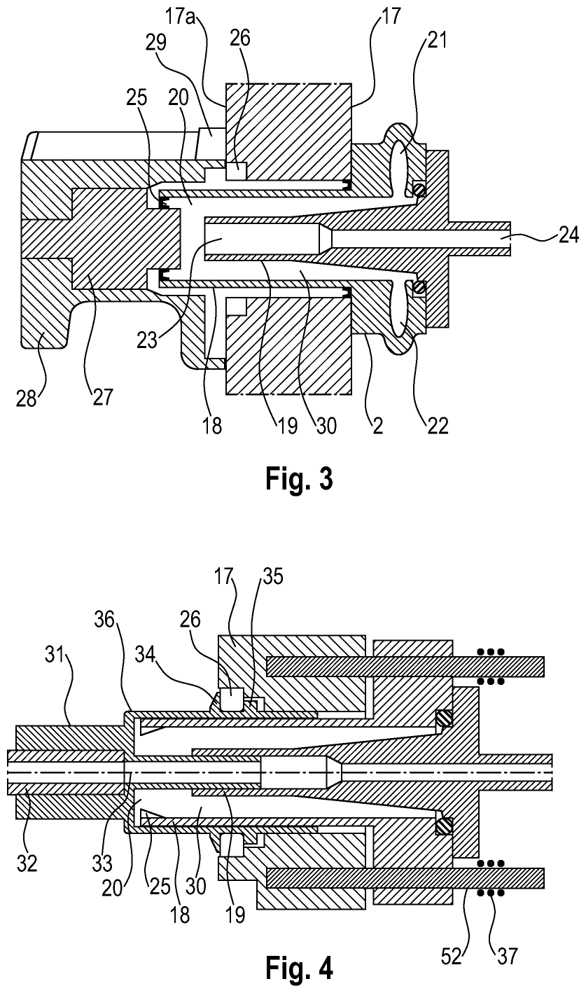

FIG. 3 shows a longitudinal section A of an embodiment of the connection unit 2, 2′ closed with the closure means 27 which is fastened in the lever handle of the pivot lever 28 and which is cylindrical and made of stainless steel. The closure means is screwed into the lever handle and can be easily replaced for maintenance. The pivot lever 28 has a peripheral moulded seal 29 on the side directed to the outer housing front 17a. The closure means 27 protrudes into the interior of the outer tube 18 of the connection unit. On the inner surface of that end of the outer tube 18 directed to the exterior of the treatment device, an elastic sealing element 29 is mounted, being firmly pressed in, which with the cylindrical element of the closure element forms a radial form-fit seal. The connection unit 2, 2′ is formed by the outer tube 18 and by the inner tube 19 arranged concentrically in the lumen of the outer tube 18. Outer tube 18 and inner tube 19 protrude through a wall of the housing 17 of the treatment machine, in which thread means 26 for fastening the disposable (not shown here) are provided on the outer appliance front 17a. The outer tube 18 protrudes through the wall out of the housing 17 of the treatment machine, such that the first outer-tube opening 20 is not arranged on a plane with the outer housing front 17a and outside the treatment machine. The second and the third outer-tube opening 21, 22 are arranged in the interior of the treatment machine. Likewise, the first inner-tube opening 23 is arranged outside the treatment machine, and the second inner-tube opening 24 is arranged in the interior of the treatment machine. The first inner-tube opening 23 is set back by 6 mm in the direction of the machine interior in relation to the first outer-tube opening 20, when the latter has an opening diameter of 1 mm, such that contacts in plug-in processes can be avoided. In the arrangement shown here, the connection unit 2, 2′ is located in the so-called short-circuit position, in which the connection unit 2, 2′ can be flushed and disinfected. For this purpose, flushing or disinfecting solution is then conveyed through the second inner-tube opening into the inner tube and from there into the outer tube. It can be discharged through the third outer-tube opening into the dialysate discharge. Before the connection unit 2, 2′ is connected to a disposable, the connection unit 2, 2′ is intended to be emptied of liquid. The emptying is effected by pumping off the liquid at the lower, third outer-tube opening 22. At the same time, sterile air is conveyed through the second outer-tube opening 21 into the lumen 30 in order to compensate for the underpressure that arises. During this emptying, the correct speed is important. In order to avoid the water column tearing off in the port, the flow rate should be less than 50 ml/min. To further optimize this process, the second outer-tube opening 21 is arranged spatially above the third outer-tube opening 22. The lumen 30 between outer tube 18 and inner tube 19 tapers in the direction of the interior of the treatment machine in order to reduce the annular gap in the rear region, together with a slight inclination of the whole component. This geometry permits optimized emptying, but also complete filling of the cavities in the connector.

FIG. 4 shows a longitudinal section B of an embodiment of the connection unit 2, 2′ connected to a plugged-in connector 31.

The whole connection unit 2, 2′ is fastened in the housing in an axially resilient manner by means of the helical springs 37. This serves for tolerance compensation of the conical plug connection of the connector 31 in the inner tube 19 of the connection unit 2, 2′. It is thus ensured that slight dimensional differences on the disposable article or on the machine components do not lead to any losses of leaktightness of the interface upon connection.

After the connector 31 has been connected to the connection unit 2, 2′, the touch-guard sleeve remains completely outside the outer tube 18 of the connection unit with the sealing element 25. This has the advantage that particles or microbes in regions inaccessible to the disinfection are not entrained from the disposable into the interior of the port. The inner tube 33 of the disposable is inserted sealingly into the inner tube 19 of the connection unit 2, 2′. The connection of the connector is secured via a bayonet fastener, which is formed between the thread means 26 in the housing 17 and cams on the touch-guard sleeve. The connector is screwed onto the connection unit. An ergonomically advantageous rotary movement of only about 30° is sufficient. The plug-in force of the cone is defined solely by the spring force of the axial spring arrangement 37, which is between 20 and 40N. This prevents a connection that is too firm and that could be problematic when disassembling the disposable.

Arranged on the outer surface of the touch-guard screen 36 is a sealing means 33, which is arranged as a peripheral lamellar wall and has been injection moulded on the touch-guard tube at a slight angle. It is conceivable for liquid to run down the housing front during a treatment. i.e. with the disposable adapter assembled. The lamella also prevents contamination and incrustation caused by liquid penetrating into the thread 26 of the connection unit.

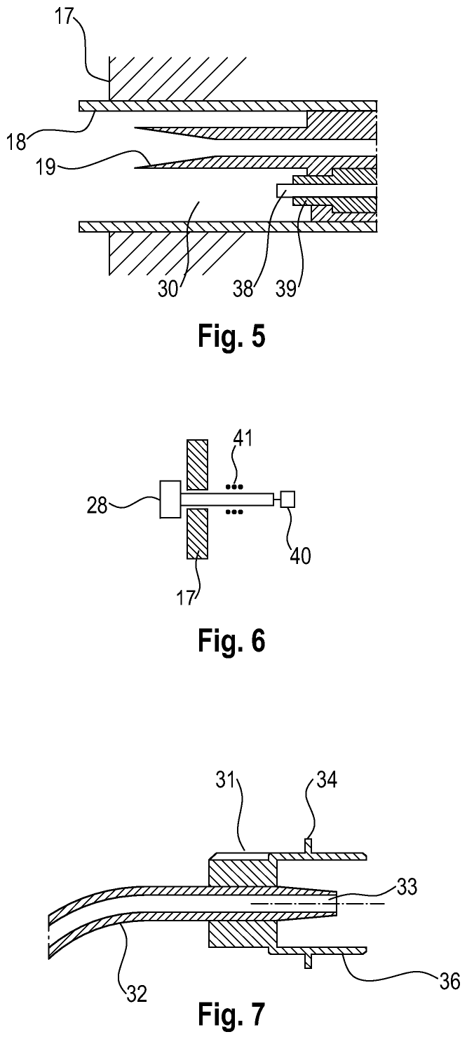

FIG. 5 shows a schematic view of the leakage sensor. To detect losses of leaktightness at the cone connection between connection unit 2, 2′ and the connector 31 (not shown), a conductivity sensor 38 is arranged in the lumen 30 between inner tube 19 and outer tube 18 of the connection unit 2, 2′. For this purpose, a galvanic isolation 39 is arranged between inner tube 19 and outer tube 18.

FIG. 6 shows a schematic view of the pivot lever 28. The pivot lever is composed of a lever handle, which is arranged outside the machine housing, and a lever axle.

To open the connection unit 2, 2′, the pivot lever 28 is pulled at the lever handle as far as an axial stop. By way of a slotted guide (not shown) at the lever axle, a rotary movement is mechanically enabled at the stop. The lever handle can be pivoted to the side only in this position. The position is dimensioned such that the lever handle can be pivoted away with sufficient spacing over the first outer-tube opening 20. The axial movement is mechanically prevented in the pivoting region. This blocking is cancelled only at the stops. When the lever handle reaches the stop position, the axial movement is released again and the lever handle is pulled against the outer housing front 17a via the spring 41. The closure of the connection unit 2, 2′ takes place similarly. The axial movement is slowed by a damping mechanism 40. This serves to ensure that any liquid residues or particles located on the closure means do not come loose and cause contamination of the interior of the connection unit 2, 2′.

FIG. 7 shows a longitudinal section through the connector 31. The connector has a conical inner tube 33 in which the sterile infusion solution is guided into the extracorporeal hose system via the fluid line. Said inner tube is enclosed by a concentrically arranged touch-guard sleeve 36. The sterile inner tube 33 is set back by 8 mm in relation to the touch-guard sleeve 36, such that it is protected from accidental contact and collision with machine surfaces. Two bevelled cams 35 are injection moulded on the outer face of the touch-guard sleeve. They serve as thread means for connection to the thread means 26 (not shown) of the machine. A peripherally arranged lamellar wall 34, which is injection moulded at a slight angle on the touch-guard sleeve, serves for sealing the connector 31 with respect to the machine front.

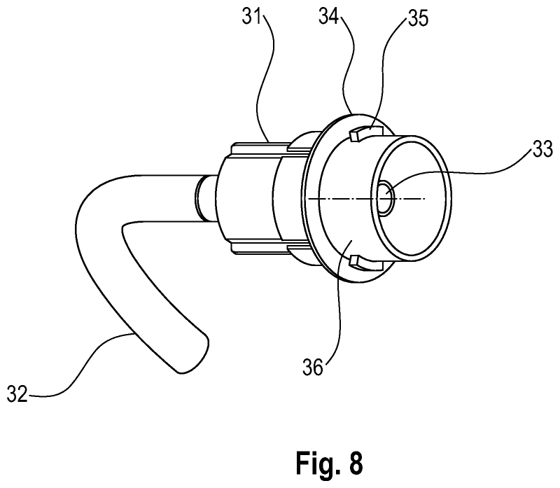

FIG. 8 shows a plan view of the connector 31, in which the cams 35 are also shown.

List of reference signs

| | |
|---|---|
| 1 | medical treatment unit |
| 2, 2′ | connection unit |
| 3 | dialysate inlet |
| 4 | dialysate discharge |
| 5 | sterile filter 1 |
| 6 | sterile filter 2 |
| 7 | air source |
| 8 | fresh dialysate coupling |
| 9 | discharge dialysate coupling |
| 10 | haemofilter |
| 11 | blood inlet line |
| 12 | blood discharge line |
| 13 | dialysate source |
| 14 | dialysate drain |
| 15 | position sensor |
| 16 | control unit |
| 17 | housing |
| 17a | outer housing front |
| 18 | outer tube of connection unit |
| 19 | inner tube of connection unit |
| 20 | first outer-tube opening |
| 21 | second outer-tube opening |
| 22 | third outer-tube opening |
| 23 | first inner-tube opening |
| 24 | second inner-tube opening |
| 25 | sealing element |
| 26 | thread means |
| 27 | closure means |
| 28 | pivot lever |
| 29 | sealing of pivot lever |
| 30 | lumen between inner tube and outer tube |
| 31 | connector |
| 32 | fluid line of disposable |
| 33 | connector lumen of inner tube of disposable |
| 34 | sealing means of disposable |
| 35 | cam |
| 36 | touch-guard sleeve/outer tube of disposable |
| 37 | fastening means/helical spring |
| 38 | leakage sensor |
| 39 | galvanic isolation |
| 40 | damping |
| 41 | resilient fastening of pivot lever |
| 49 | semipermeable membrane |
| 50 | blood chamber |
| 51 | dialysis liquid chamber |
| 52 | fastening means |

1. A medical treatment device with a housing and with a connection unit that protrudes through a wall of the housing for producing a fluidic connection between at least a first fluid line assigned to the interior of a treatment device and a connector connected to a second fluid line;
wherein the connection unit has an outer tube
and an inner tube arranged in the lumen of the outer tube,
the outer tube has a first outer-tube opening at the end facing the exterior of the treatment device and at least a second and third outer-tube opening at the end facing the interior of the treatment device,
the inner tube has a first inner-tube opening at the end facing the exterior of the treatment device and at least a second inner-tube opening at the end facing the interior of a treatment device, wherein
the outer tube protrudes permanently out of the housing.

2. The medical treatment device according to claim 1, wherein the distance between the wall of the housing and the first outer-tube opening of the connection unit measures at least 10 mm.

3. The medical treatment device according to claim 1, wherein the lumen between outer tube and inner tube tapers conically in the direction of the interior of the treatment device.

4. The medical treatment device according to claim 1, wherein the connection unit, is fastened in an axially resilient manner to the housing of the machine by a fastening means.

5. The medical treatment device according to claim 4, wherein the fastening means is a helical spring.

6. The medical treatment device according to claim 1, wherein an elastic sealing element is mounted on the inner surface of that end of the outer tube directed to the exterior of the treatment device.

7. The medical treatment device according to claim 1, wherein a leakage sensor is arranged in the connection unit, in the lumen between outer tube and inner tube.

8. The medical treatment device according to claim 1, wherein the treatment device has a closure element for closing that end of the outer tube directed to the machine exterior.

9. The medical treatment device according to claim 8, wherein the closure element is a cylindrical element which in the closure state protrudes into the outer tube of the connection unit and there forms a radial form-fit seal.

10. The medical treatment device according to claim 8, wherein the closure element is provided in a receiving portion of a pivot lever which, for opening and closing the connection unit, is displaceable in translation along a pivot axis, and wherein the treatment device has a damping device for slowing this translational movement.

11. A disposable comprising a fluid line and a connector with a connector lumen for fluidically connecting the fluid line to the inner tube of a connection unit and the medical treatment device according to claim 1, and the connector has a touch-guard sleeve with a free connection opening and a wall, and the connector lumen, surrounded by the wall, leads into an opening that is set back in relation to the free connection opening of the touch-guard sleeve, wherein the outer surface of the touch-guard sleeve has a radially outwardly directed sealing means.

12. The disposable according to claim 11, wherein the touch-guard sleeve has, on the outer surface, means for producing a threaded closure.

13. A system comprising the medical treatment device according to claim 1 and a disposable comprising a fluid line and a connector with a connector lumen for fluidically connecting the fluid line to the inner tube of a connection unit and the medical treatment device, and the connector has a touch-guard sleeve with a free connection opening and a wall, and the connector lumen, surrounded by the wall, leads into an opening that is set back in relation to the free connection opening of the touch-guard sleeve, wherein the outer surface of the touch-guard sleeve has a radially outwardly directed sealing means.

14. The system according to claim 13, wherein the outer tube of the connection unit is inserted into the lumen of the touch-guard sleeve, and the connector lumen of the disposable is inserted into the lumen of the inner tube of the connection unit.

15. The system according to claim 13, wherein the connection of disposable and attachment site is secured by thread means on the outer surface of the touch-guard sleeve and on the housing of the treatment device.

16. The medical treatment device according to claim 1, wherein the outer tube, is fastened in an axially resilient manner to the housing of the machine by a fastening means.

17. The medical treatment device according to claim 1, wherein an elastic sealing element is mounted on the inner surface of that end of the outer tube directed to the exterior of the treatment device and being pressed firmly into the outer tube.

18. The medical treatment device according to claim 1, wherein a conductivity sensor, is arranged in the connection unit, in the lumen between outer tube and inner tube.

19. The medical treatment device according to claim 8, wherein the closure element is a cylindrical element of stainless steel, which in the closure state protrudes into the outer tube of the connection unit and there forms a radial form-fit seal.

20. The medical treatment device according to claim 8, wherein the closure element is provided in a receiving portion of a pivot lever which, for opening and closing the connection unit, is displaceable in translation along a pivot axis, and arranged perpendicular to the housing surface, and wherein the treatment device has a damping device for slowing this translational movement.
